(19) United States
(12) Patent Application Publication
(10) Pub. No.: US 2017/0182080 A1
(43) Pub. Date: Jun. 29, 2017

(54) FUNCTIONAL SCAFFOLD FOR TISSUE REPAIR AND REGENERATION

(71) Applicant: Bo HAN, Temple City, CA (US)

(72) Inventor: Bo HAN, Temple City, CA (US)

(21) Appl. No.: 15/126,133

(22) PCT Filed: Mar. 16, 2015
(86) PCT No.: PCT/US15/20648

§ 371 Date: Sep. 14, 2016

Related U.S. Application Data
(60) Provisional application No. 61/953,589, filed on Mar. 14, 2014.

(51) Int. Cl. A61K 31/7068; A61L 27/14; A61L 27/10; A61L 27/54; A61L 27/04

(52) U.S. Cl. CPC A61K 31/7068; A61L 27/54; A61L 27/04; A61L 27/10; A61L 27/14; A61L 2300/414; A61L 2300/252; A61L 2300/426; A61L 2300/258

(57) ABSTRACT

A biocompatible scaffold is provided that includes a biocompatible scaffold substrate and an amount of a demethylation agent or a methylation inhibitor, in an amount effective to induce and/or promote tissue repair and regeneration. The demethylation agent or methylation inhibitor can be a cytidine analog. The scaffold can further include an effective amount of one or more of a target specific growth factor, a cytokine, a signaling molecule or a pharmaceutically acceptable carrier. The scaffold can be constructed of a natural or non-natural material; one alternative is a gel formed by the catalytic activity of transglutaminase.

### CROSS-REFERENCE TO RELATED APPLICATION

This application claims the benefit of U.S. Provisional Application Ser. No. 61/953,589 by Dr. Bo Han, entitled “Functional Scaffold for Tissue Repair and Regeneration,” and filed Mar. 14, 2014, the contents of which are incorporated herein in their entirety by this reference.

### FIELD OF THE INVENTION

This invention is directed to functional scaffolds for tissue repair and regeneration comprising a biocompatible scaffold substrate and a demethylation agent or methylation inhibitor, and methods for the use of such functional scaffolds for tissue repair and regeneration.

### BACKGROUND OF THE INVENTION

In order for tissues to repair or regenerate, cells must first migrate into a wound bed, proliferate, express matrix components or form extracellular matrix, and then form a final tissue shape. This process involves a variety of cell populations interacting in an intricate web of cascading events. To facilitate this process, successful artificial regeneration of tissues usually requires a physical scaffold for cell migration and proliferation.

Prior art scaffolds are complex systems. A need exists in the art for a biocompatible system; elegant in its simplicity and effectiveness. This invention satisfies this need and provides related advantages as well.

### SUMMARY OF THE INVENTION

This disclosure provides a biocompatible scaffold, comprising, or alternatively consisting essentially of, or yet further consisting of, biocompatible scaffold substrate and an amount of a demethylation agent or a methylation inhibitor, in an amount effective to induce and/or promote tissue repair and regeneration. In one aspect, the methylation inhibitor comprises a cytidine analogue, e.g., one or more of 5-azacytidine, 5-aza-2′-deoxycytidine, peudoisocytidine, or 5-fluoro-2′-deoxycytidine.

The scaffold can further comprise an effective amount of one or more of a target specific growth factor, a cytokine, a signaling molecule or a pharmaceutically acceptable carrier.

In one aspect, the biocompatible scaffold treats a subject upon implantation into a specific site requiring treatment in the subject. The subject is an animal, a mammal (feline, canine, bovine or equine, for example) or a human patient. For example, the scaffold has been shown herein to promote tissue regeneration in a subject in need thereof.

Kits for preparing the methods and use of the materials are further provided herein.

### BRIEF DESCRIPTION OF THE DRAWINGS

The following invention will become better understood with reference to the specification, appended claims, and accompanying drawings, where:

FIG. 1 is a graph showing results of cell proliferation using an antibody specifically binding the proliferation marker Ki-67, indicating the effectiveness of Aza in activating quiescent cells.

FIG. 2 is a graph showing the effect of AZA-CR in enhancing alkaline phosphatase (ALP) activity, showing osteogenic differentiation.

FIG. 3 is a graph showing the effect of AZA-CR in enhancing calcium deposition, showing osteogenic differentiation.

FIG. 4 is a graph showing the effect of AZA-CR in enhancing the expression of the myogenic marker MyoD in adipose cell cultures.

FIG. 5 is a graph showing the effect of AZA-CR in enhancing the expression of N-cadherin in MSC or adipose cells treated with AZA-CR.

FIG. 6 is a graph showing the effect of AZA-CR in enhancing adipogenesis as demonstrated by Oil Red O staining.

FIG. 7 shows the results of chondrogenic cell regeneration after treatment with AZA-CR (4.5% Tg-Gel, control, 7 days, left panel; 4.5% T-Gel +250 nmol Aza, 7 days, right panel).

FIG. 8 shows the results of treatment with AZA-CR in inducing ectopic bone formation in young animals with staining with hematoxylin and eosin (H&E) for visualization of cells and tissue formation (8A: collagen; 8B: Collagen+100 ng; 8C: Collagen+100 ng+50 nM Aza-CR; 8D: Collagen+100 ng+500 nM Aza-CR).

FIG. 9 shows the results of treatment with AZA-CR in inducing ectopic bone formation in young animals by assay of alkaline phosphatase.

FIG. 10 shows the results of treatment with AZA-CR in inducing ectopic bone formation in aged animals with staining with hematoxylin and eosin (H&E) for visualization of cells and tissue formation.

FIG. 11 shows the results of treatment with AZA-CR in inducing bone formation at a bony defect site staining with hematoxylin and eosin (H&E) for visualization of cells and tissue formation.

FIG. 12 shows the results of treatment with AZA-CR in inducing spinal fusion after introduction of spinal lesions in terms of radiographic assessment and histology.

FIG. 13 shows the results of treatment with AZA-CR in inducing muscle regeneration; the top panels show the results of H&E staining for morphology and the bottom panels show the results of OCT-4 IHC staining for immunological specific indication of stem cell growth; the left panels are without AZA-CR and the right panels are with AZA-CR.

FIG. 14 shows the results of treatment with AZA-CR in inducing cartilage repair; the left panel shows the 4.5% Tg-gel control at 7 days and the right panel the 4.5% Tg-gel +250 nmole Aza at 7 days.

### DETAILED DESCRIPTION OF THE INVENTION

Throughout this application, the text refers to various embodiments of the present compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention. Also throughout this disclosure, various publications, patents and published patent specifications are referenced by an identifying citation. The disclosures of these publications, patents and published patent specifications are hereby incorporated by reference into the present disclosure in their entirety to more fully describe the state of the art to which this invention pertains.

### DEFINITIONS

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, a Laboratory Manual; and Animal Cell Culture (R. I. Freshney, ed. (1987)).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a cell” includes a plurality of cells, including mixtures thereof, unless otherwise excluded.

The phrase “equivalent protein” refers to protein or polynucleotide which hybridizes to the exemplified polynucleotide under stringent conditions and which exhibit similar or enhanced biological activity in vivo, e.g., over 120%, or alternatively over 110%, or alternatively over 100%, or alternatively, over 90% or alternatively over 85% or alternatively over 80%, or alternatively over 70%, as compared to the standard or control biological activity. Additional embodiments within the scope of this invention are identified by having more than 80%, or alternatively, more than 85%, or alternatively, more than 90%, or alternatively, more than 95%, or alternatively more than 97%, or alternatively, more than 98 or 99% sequence identity. Percentage identity can be determined by sequence comparison programs such as BLAST run under appropriate conditions. In one aspect, the program is run under default parameters.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double-and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

A “gene” refers to a polynucleotide containing at least one open reading frame (ORF) that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art.

The terms “express,” “expression,” or similar terminology refer to the production of a gene product.

Hybridization reactions can be performed under conditions of different “stringency”. In general, a low stringency hybridization reaction is carried out at about 40 ⁰C in 10×SSC or a solution of equivalent ionic strength/temperature. A moderate stringency hybridization is typically performed at about 50° C. in 6×SSC, and a high stringency hybridization reaction is generally performed at about 60° C. in 1 x SSC.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described in Current Protocols in Molecular Biology (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code =standard; filter =none; strand =both; cutoff =60; expect =10; Matrix =BLOSUM62; Descriptions =50 sequences; sort by =HIGH SCORE; Databases =non-redundant, GenBank+EMBL+DDBJ+PDB+GenBank CDS translations+SwissProtein+SPupdate+PIR. Details of these programs can be found at the following Internet address:

http://www.ncbi.nlm.nih.gov/cgi-bin/BLAST.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, or other covalent bonds known in the art. As used herein the term “amino acid” refers to either natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein.

As used herein, the term “comprising” is intended to mean that the compositions and methods include the recited elements, but not excluding others. “Consisting essentially of ” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “isolated” means separated from constituents, cellular and otherwise, in which the cell, tissue, polynucleotide, peptide, polypeptide, protein, antibody or fragment(s) thereof, which are normally associated in nature. For example, an isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. An isolated cell is a cell that is separated form tissue or cells of dissimilar phenotype or genotype.

The term “propagate” means to grow or alter the phenotype of a cell or population of cells. The term “growing” or “expanding” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue.

The term “culturing” refers to the in vitro propagation of cells or organisms on or in media of various kinds It is understood that the descendants of a cell grown in culture may not be completely identical (i.e., morphologically, genetically, or phenotypically) to the parent cell. By “expanded” is meant any proliferation or division of cells.

“Clonal proliferation” refers to the growth of a population of cells by the continuous division of single cells into two identical daughter cells and/or population of identical cells.

As used herein, the “lineage” of a cell defines the heredity of the cell, i.e. its predecessors and progeny. The lineage of a cell places the cell within a hereditary scheme of development and differentiation.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type or phenotype. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

Examples of cells that differentiate into endodermal lineage include, but are not limited to pleurogenic cells, and hepatogenic cells, cells that give rise to the lining of the intestine, and cells that give rise to pancreogenic and splanchnogenic cells.

As used herein, a “pluripotent cell” defines a less differentiated cell that can give rise to at least two distinct (genotypically and/or phenotypically) further differentiated progeny cells.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker, e.g. myosin or actin or the expression of a gene or protein.

“Bone Morphogenic Proteins” (BMP) are a group of multifunctional growth factors and cytokines with effects in various tissues. For example, BMPs are known to induce the formation of bone and/or cartilage. Examples of BMP may include, but are not limited to BMP1, BMP2, BMP3, BMP4, BMP5, BMP6, BMP7, BMP8a, BMP8b, BMP10 and BMP15.

“BMP signaling” or “BMP signaling pathway” refers to the enzyme linked receptor protein signaling transduction pathway involving proteins that directly or indirectly regulate (activate or inhibit) downstream protein activity or gene expression. Examples of molecules involved in the BMP signaling pathways may be found in the public Gene Ontology (GO) database, under GO ID: GO:0030509, accessible at the web page (amigo.geneontology.org/cgi-bin/amigo/term-details.cgi?term=G0:0030509&sessionid=5573amigo1226631957), last accessed on Nov. 17, 2008. Without limitation, examples of proteins in the BMP signaling pathway include Activin receptor type-1 (ACVR1, UniProt: Q04771), Activin receptor type-2A (ACVR2A, UniProt: P27037), Activin receptor type-2B (ACVR2B, UniProt: Q13705), BMP1 (UniProt: P13497), BMP2 (UniProt: P12643), BMP3 (UniProt: P12645), BMP4 (UniProt: P12644), BMP5 (UniProt: P22003), BMP6 (UniProt: P22004), BMP7 (UniProt: P18075), BMP8a (UniProt: Q7Z5Y6), BMP8b (UniProt: P34820), BMP10 (UniProt: 095393), BMP15 (UniProt: 095972), Bone morphogenetic protein receptor type-1A (BMPR1A, UniProt: P36894), Bone morphogenetic protein receptor type-1B (BMPR1B, UniProt: 000238), Bone morphogenetic protein receptor type-2 (BMPR2, UniProt: Q13873), Chordin-like protein (CHRDL1, UniProt: Q9BU40), Follistatin-related protein 1 (FSTL1, UniProt: Q12841), Growth/differentiation factor 2 (GDF2, UniProt: Q9UK05), Growth/differentiation factor 6 (GDF6, UniProt: Q6KF10), Growth/differentiation factor 7 (GDF7, UniProt: Q7Z4P5), Gremlin-2 (GREM2, UniProt: Q9H772), RGM domain family member B (RGMB, UniProt: Q6NW40), Ski oncogene (SKI, UniProt: P12755), Mothers against decapentaplegic homolog 4 (SMAD4, UniProt: Q13485), Mothers against decapentaplegic homolog 5 (SMADS, UniProt: Q99717), Mothers against decapentaplegic homolog 6 (SMAD6, UniProt: 043541), Mothers against decapentaplegic homolog 7 (SMAD7, UniProt: 015105), Mothers against decapentaplegic homolog 9 (SMAD9, UniProt: 015198), E3 ubiquitin-protein ligase SMRF2 (SMURF2, UniProt: Q9HAU4), TGF-beta receptor type III (TGFBR3, UniProt: Q03167), Ubiquitin-conjugating enzyme E2 DI (UBE2D1, UniProt: P51668), Ubiquitin-conjugating enzyme E2 D3 (UBE2D3, UniProt: P61077) and Zinc finger FYVE domain-containing protein 16 (ZFYVE16, UniProt: Q7Z3T8). Proteins that positively or negatively regulate the BMP signaling, for purpose of this invention, are also considered within the meaning of the BMP signaling. Proteins that positively regulate BMP signaling include, but are not limited to, Serine/threonine-protein kinase receptor R3 (ACVRL1, UniProt: P37023) and Endoglin (ENG, UniProt: P17813). Proteins that negatively regulate BMP signaling include, but are not limited to, Chordin (CHRD, UniProt: Q9H2X0), E3 ubiquitin-protein ligase SMURF1 (SMURF1, UniProt: Q9HCE7), Sclerostin (SOST, UniProt: Q9BQB4) and Brorin (VWC2, UniProt: Q2TAL6). Examples of proteins in the BMP signaling pathway may also include Proprotein convertase subtilisin/kexin type 6 (PCSK6, UniProt: P29122) that regulates BMP signaling.

Other types of BMP agonists or antagonists also exist. Yanagita (2009) BioFactors 35(2):113-199 is a review article discussing BMP regulators (incorporated by reference). Non-limiting examples include such as noggin, chordin, gremlin, sclerostin and follistatin. Representative sequences for these proteins include UniProt: Q13253 for noggin, UniProt: Q9H2X0 for chordin, UniProt: 060565 for gremlin, UniProt: Q9BQB4 for sclerostin, and UniProt: P19883 for follistatin. Noggin (UniProt: Q13253), for example, can be produced using methods described in, e.g. McMahon et al. (1998) Genes & Development 12:1438-52.

“Transforming growth factor-β” (TGF-β) is a ubiquitously expressed, secreted pleiotropic cytokine that exists in mammals in three isoforms TGF-β1 (UniProt: P01137), TGF-β2 (UniProt: P61812), and TGF-β3 (UniProt: P10600). All three TGF-β isoforms interact with the same high-affinity receptors which include type I (TGF-(βRI, or ALK-5, UniProt: P36897), type II (TGF-(βRII, UniProt: P37173), and type III (TGF-βRIII, or betaglycan, UniProt: Q03167). The TGF-βRI and TGF-βRII receptors are signaling receptors and contain serine-threonine protein kinases in their intracellular domains that initiate intracellular signaling by phosphorylating transcription factors from the SMAD pathway; in contrast, the TGF-βRIII receptor is the only nonsignaling, but most abundant receptor (Blobe G C et al., 2000 N Engl J Med 342:1350-1358).

TGF-β is produced in a latent form consisting of TGF-β and the non-covalently bound latency-associated peptide, LAP, derived from the N-terminal of the TGF-β precursor, and must be released for activation.

TGF-βRI is a protein of 503 amino acids that contains a signal sequence and cysteine-rich N-terminal extracellular domain followed by a transmembrane domain and a cytoplasmic serine, threonine kinase domain (Franzen, P. et al., 1993 Cell 75, 681-692). The extracellular domain has little sequence similarity with the TGF-βRII receptor, but the cytoplasmic domain has more with respect to that of the TGF-βII. In the cytoplasmic domain, eleven major conserved subdomains are evident, including the ATP-binding site and the catalytic domain (Hanks, S. K., Quinn, A. M. and Hunter, T. 1988 Science 241, 42-52).

TGF-βRII is a 565 amino acid protein with a signal sequence and cysteine-rich N-terminal extracellular domain followed by a transmembrane domain and a cytoplasmic serine, threonine kinase domain (Lin, H. Y. et al., 1992 Cell 68, 775-785).

TGF-βRIII is a protein of 853 amino acids that contains a signal sequence and large N-terminal extracellular domain followed by a transmembrane domain and a short cytoplasmic tail of 41 amino acids (Wang, X.-F. et al., 1991 Cell 67, 797-805).

“Growth differentiation factor” (GDF) belongs to the transforming growth factor beta superfamily, including but not limited to GDF1 (UniProt: P27539), GDF10 (UniProt: P55107), GDF11 (UniProt: 095390), GDF15 (UniProt: Q99988), GDF2 (also known as BMP-9, UniProt: Q9UK05), GDF3 (also known as Vgr2, UniProt: Q9NR23), GDF3A (UniProt: Q8NI58), GDFS (also known as CDMP-1 or MP52, UniProt: P43026), GDF6 (also known as CDMP-2, UniProt: Q6KF10), GDF7 (also known as CDMP-3, UniProt: Q7Z4P5), GDF8 (as known as MSTN, UniProt: 014793), and GDF9 (UniProt: 060383).

“Glial cell line-derived neurotrophic factor” (GDNF) family ligands (GFLs) are distant members of the TGF-β superfamily that are potent neurotrophic factors in vitro and are critical for the development of distinct neuronal populations. There are four known members of this family with high sequence similarity; GDNF (UniProt: P39905), neurturin (NRTN, UniProt: Q99748), (ARTN, UniProt: Q5T4W7) and (PSPN, UniProt: 060542). These factors all function through the activation of the transmembrane receptor tyrosine kinase rearranged during transfection (RET), which activates multiple signaling pathways. “Glial cell-derived neurotrophic factor” (GDNF) is a protein that promotes the survival of various types of neurons. “Neurturin” (NRTN) is a ligand used to bind to GFRA2 receptors. “Artemin” (ARTN) is also known as enovin or neublastin. (Airaksinen, M. S., et al., (1999) “GDNF family neurotrophic factor signaling: four masters, one servant?” Mol. Cell. Neurosci. 13, 313-325; Knowles, P P. (2006) “Structure and chemical inhibition of the RET tyrosine kinase domain”, J. Biol. Chem. 281 33577-33587).

An “inhibin” (INH) is a heterodimeric molecule containing an a subunit (INHA, UniProt: P05111) and either an I3A (INHBA, UniProt: P08476) or I3B(INHBB, UniProt. P09529) subunit, which are connected to each other by a disulfide bond. If the dimer consists of an I3A subunit the molecule is called inhibin A, and if it consists of an I3B subunit the molecule is called inhibin B. The inhibin can be from a human or non-human animal (e.g., horse, bovine, goat, dog, cat, sheep, rabbit, mouse, rat, non-human primate, manatee, or other animal species), and inter-species sequence conservation is relatively high. An exemplary human alpha subunit sequence is disclosed as SwissProt Accession No. P05111.1. Exemplary human beta-A and beta-B subunit sequences are disclosed as SwissProt Accession Nos. P08476.2 and P09529.2, respectively.

The terms “activin beta-B subunit,” “inhibin beta-B subunit,” “inhibin B beta subunit,” and “inhibin/activin beta-B subunit” refer to the same subunit polypeptide. Similarly, the terms “activin beta-A subunit,” “inhibin beta-A subunit,” “inhibin A beta subunit,” and “inhibin/activin beta-A subunit” refer to the same subunit polypeptide.

An “activin” contains two 13 subunits and can be homodimeric or heterodimeric depending on the arrangement of their subunits. Two I3A subunits make activin A, two I3B subunits make activin B and an I3A subunit attached to a PB subunit make activin AB. The activin can be from a human or non-human animal, e.g., horse, bovine, goat, dog, cat, sheep, rabbit, mouse, rat, non-human primate, or other animal species.

Activins and inhibins can play opposing roles in diverse systems, including hypothalamic and pituitary hormone secretion, gonadal hormone secretion, germ cell development and maturation, erythroid differentiation, insulin secretion, nerve cell survival, embryonic axial development, and bone growth, depending on subunit composition.

“Left-Right Determination Factor” (LEFTY) refers to proteins that are closely related members of the TGF-β family of growth factors. These proteins are secreted and play a role in left-right asymmetry determination of organ systems during development, including but not limited to LEFTY1 (UniProt: 075610) and LEFTY2 (UniProt: 000292). (Hamada H, et al., 2002, Establishment of vertebrate left-right asymmetry. Nat. Rev. Genet. 3 (2): 103-13).

“Myostatin” (MSTN, also known as growth differentiation factor 8, abbreviated GDF-8, UniProt. 014793) is a secreted growth differentiation factor that is a member of the TGF beta protein family that inhibits muscle differentiation and growth in the process known as myogenesis. (Gonzalez-Cadavid N F, et al., 1998, Organization of the human myostatin gene and expression in healthy men and HIV-infected men with muscle wasting, Proc. Natl. Acad. Sci. U.S.A. 95 (25): 14938-43).

“Nodal” (NODAL, UniProt: Q96S42) is a secretory protein that belongs to the Transforming Growth Factor (TGF-beta) superfamily. It is involved in cell differentiation in early embryogenesis, playing a key role in signal transfer from the node, in the anterior primitive streak, to lateral plate mesoderm. (Gebbia M, Ferrero G B, et al., 1997, X-linked situs abnormalities result from mutations in ZIC3, Nat Genet 17 (3): 305-8).

“Anti-Müllerian hormone” (AMH, UniProt: P03971), also known as Müllerian inhibiting substance (MIS) or Müllerian inhibiting factor (MIF), is a member of the transforming growth factor β (TGF-β) superfamily of peptide growth and differentiation factors. (Cate R L, et al., 1986, Isolation of the bovine and human genes for Miillerian inhibiting substance and expression of the human gene in animal cells, Cell 45 (5): 685-98).

“Fibroblast Growth Factors” (FGFs) are classified as acidic (aFGF) or basic (bFGF) depending on their isoelectric points, including but are not limited to FGF1 (UniProt: P05230) and FGF2 (UniProt: P09038). FGFs are a family of polypeptides (Ginineq-Gallego et al., Biochem. Biophys. Res. Commun., 1986, 135: 541-48; Thomas et al., Trends Biochem. Sci., 1986, 11:81-84) or derivatives thereof, obtained from natural, synthetic or recombinant sources, which exhibit the ability to stimulate DNA synthesis and cell division in vitro in a variety of cells, including primary fibroblasts, chondrocytes, vascular and coreal, and glial cells (Canalis et al., Clin. Invest., 1988, 81:1572-77).

“Platelet-derived growth factor” (PDGF) is a protein that regulates cell growth and division and plays a significant role in blood vessel formation (angiogenesis), the growth of blood vessels from already-existing blood vessel tissue. PDGF is a dimeric glycoprotein composed of two A (−AA) or two B (−BB) chains or a combination of the two (−AB). Exemplary human PDGF subunits A and B are disclosed as UniProt Entry Nos. P04085 and P01127, respectively.

“Vascular endothelial growth factor” (VEGF, UniProt: P15692) is a protein produced by cells that stimulates vasculogenesis and angiogenesis. VEGF functions to restore the oxygen supply to tissues when blood circulation is inadequate. VEGF can create new blood vessels during embryonic development and after injury.

“Insulin-like growth factors” (IGFs) are proteins with high sequence similarity to insulin. These peptides are useful in treating disorders caused or mediated by IGFs, such as cancer. IGFs include but not limited to IGF-1 (UniProt: P05019), IGF-2 (UniProt: P01344), and IGF variants. Human IGF-1 belongs to a family of somatomedins with insulin-like and mitogenic biological activities that modulate the action of growth hormone. IGFs are structurally similar to insulin, and have been implicated in a variety of cellular functions and disease processes. The IGF system is also composed of membrane-bound receptors for IGF-1, IGF-2, and insulin. The Type 1 IGF receptor (IGF-IR, UniProt: P08069) is closely related to the insulin receptor in structure and shares some of its signaling pathways. The IGF-2 (UniProt: P11717) receptor is a clearance receptor that appears not to transmit an intracellular signal. Since IGF-1 and IGF-2 bind to IGF-1 R with a much higher affinity than to the insulin receptor, it is most likely that most of the effects of IGF-1 and IGF-2 are mediated by IGF-IR. IGF-IR is a key factor in normal cell growth and development.

“Insulin” (UniProt: P01308) is a peptide hormone, produced by beta cells in the pancreas. Insulin regulates carbohydrate and fat metabolism in the body.

“Nerve growth factor” (NGF) is a protein that plays a role in the growth, maintenance, and survival of neurons. NGFs are also known as neurotrophins, including but not limited to Brain-Derived Neurotrophic Factor (BDNF, UniProt: P23560), Neurotrophin-3 (NTF-3, UniProt: P20783), and Neurotrophin ⅘ (NTF-⅘, UniProt: P34130).

“Cytokines” are a broad and loose category of small proteins (˜5-20 kDa) that are important in cell signaling. they are released by cells and affect the behavior of other cells, and sometimes the releasing cell itself. Cytokines include chemokines, interferons, interleukins, lymphokines, and tumor necrosis factor but generally not hormones or growth factors. Cytokines are produced by broad range of cells, including immune cells like macrophages, B lymphocytes and T lymphocytes, mast cells, as well as endothelial cells, fibroblasts, and various stromal cells; a given cytokine may be produced by more than one type of cell. They act through receptors, and are especially important in the immune system; cytokines modulate the balance between humoral and cell-based immune responses, and they regulate the maturation, growth, and responsiveness of particular cell populations. Some cytokines enhance or inhibit the action of other cytokines in complex ways.

“Signaling molecules” or cell signaling is part of a complex system of communication that governs basic cellular activities and coordinates cell actions. The ability of cells to perceive and correctly respond to their microenvironment is the basis of development, tissue repair, and immunity as well as normal tissue homeostasis. Signaling molecules can belong to several chemical classes: lipids, phospholipids, amino acids, monoamines, proteins, glycoproteins, nucleotides, and gases.

A “biocompatible scaffold, matrix, sponge or support” refers to a scaffold or matrix for tissue-engineering purposes with the ability to perform as a substrate that will support the appropriate cellular activity to generate the desired tissue, including the facilitation of molecular and mechanical signaling systems, without eliciting any undesirable effect in those cells or inducing any undesirable local or systemic responses in the eventual host. In other embodiments, a biocompatible scaffold is a precursor to an implantable device which has the ability to perform its intended function, with the desired degree of incorporation in the host, without eliciting an undesirable local or systemic effects in the host. Biocompatible scaffolds are described in U.S. Patent Publ. Nos. 2010/0260845 and 2009/0291116.

For the purpose of illustration only, examples of biocompatible scaffolds for use in this disclosure include, but are not limited to the porous and/or biodegradable and/or biocompatible scaffold as described in U.S. Pat. No. 4,947,840, col. 2, line 27 to col. 5, line 10, incorporated herein by reference in its entirety. In some other embodiments, a biocompatible scaffold is a dermal substitute consisting of amnion and biodegradable polymer as described in U.S. Patent Application Publication No. US 2005/0107876, paragraphs 28 to 64. In some other embodiments, a biocompatible scaffold is a single or double density biopolymer foam as described in International Patent Application Publication No. WO 98/22154, page 5, line 32 to page 23, line 33. In some other embodiments, a biocompatible scaffold is a gel-matrix-cells integrated system as described in International Patent Application Publication No. WO 2007/141028, page 13, line 1 to page 21, line 2. In some other embodiments, a biocompatible scaffold is a biomechanical implant as described in International Patent Application Publication No. WO 98/40111, page 7, line 13 to page 19, line 9.

In some embodiments, a biocompatible scaffold is a biocompatible nanofiber matrix as described in Venugopal et al. (2005) Tissue Engineering 11(⅚):847-54.

Examples of commercially available biocompatible scaffolds include, but are not limited to, Alloderm dermal collagen matrix (LifeCell Corporation, Branchburg, N.J.), Dermagraft-TC woven bioabsorbable polymer (polyglycolic and polylactic acids) membrane (Advanced Tissue Sciences, La Jolla, Calif.), Dermalogen human dermal collagen matrix (Collagenesis, Beverly, Mass.), Integra Bilayer Matrix Wound Dressing (Integra Life Sciences Corporation, Plainsboro, N.J.) and Fibrin Sealant Tisseel VH fibrin glue mixture (Baxter Health, Deerfield, Ill.). Such scaffolds can be used in the present invention. In some embodiments, the biocompatible scaffold can be type I collagen or silicon cell culture insert which are commercially available (e.g. Falcon™ Cell Culture Insert from BD Biosciences, San Jose, Calif.).

A “composition” is intended to mean a combination of active agent, cell or population of cells and another compound or composition, inert (for example, a detectable agent or label) or active, such as a biocompatible matrix or scaffold.

A “pharmaceutical composition” is intended to include the combination of an active agent with a carrier, inert or active such as a biocompatible scaffold, making the composition suitable for diagnostic or therapeutic use in vitro, in vivo or ex vivo. Additionally, a pharmaceutical composition can include other excipients or agents conventionally employed in such compositions, and can also include at least one therapeutically active agent.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)).

An “effective amount” is an amount sufficient to effect beneficial or desired results. An effective amount can be administered in one or more administrations, applications or dosages.

A “subject,” “individual” or “patient” is used interchangeably herein, and refers to a vertebrate, preferably a mammal, more preferably a human. Mammals include, but are not limited to, murines, rats, simians, bovines, canines, humans, farm animals, sport animals and pets.

A “control” is an alternative subject or sample used in an experiment for comparison purposes. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype).

As used herein, the terms “treating,” “treatment” and the like are used herein to mean obtaining a desired pharmacologic and/or physiologic effect. The effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder, e.g., cardiac arrhythmia. As is understood by those skilled in the art, “treatment” can include systemic amelioration of the symptoms associated with the pathology and/or a delay in onset of symptoms such as chest pain. Clinical and sub-clinical evidence of “treatment” will vary with the pathology, the individual and the treatment. However, the use of terms such as “treatment” is not to be understood as implying a complete cure that would lead to cessation of treatment unless explicitly specified.

### MODES FOR CARRYING OUT THE DISCLOSURE

### Biocompatible Scaffolds

Unexpectedly, Applicant has discovered that the biocompatible scaffold compositions disclosed herein are useful for tissue repair and regeneration even in the absence of the addition of exogenously added cells or tissue. The scaffold provides an effective cell therapy without the delivery of exogenous cells to the site of repair or regeneration.

To that end, this disclosure provides a biocompatible scaffold, comprising, or alternatively consisting essentially of, or yet further consisting of, a biocompatible scaffold substrate and an amount of a demethylation agent or a methylation inhibitor, wherein the amount of the demethylation agent or methylation inhibitor is effective to induce and/or promote tissue regeneration or repair. In one aspect, the scaffold comprises, or consists essentially of, or yet further consists of, one or more of a tissue specific growth factor, a cytokine, a signaling molecule or a pharmaceutically acceptable carrier or excipient. Non-limiting examples of signaling molecules include non-protein molecules such as genes, lipids, microRNA and exosomes.

In another aspect, the biocompatible scaffold comprises one or more of calcium triphosphate, demineralized bone, cellulose, wherein the matrix conforms substantially to its insertion site and provides a structurally stable, three dimensional surface for retaining the transplant and supporting ingrowth of cells and tissue, wherein the tissue is one or more of connective tissue, bone, dermal tissue, neuronal tissue, endothelial tissue, muscle, cardiac muscle, dentin, ocular tissue and organ tissue. The tissue to be repaired or regenerated includes without limitation, muscle, bone, tendons, vascular, epithelial, endothelial, dermal, corneal, retinal, dental, connective, neuronal, facial, cranial, soft tissue including cartilage and collagen, liver, kidney, spine, central nervous system, spine, brain, peripheral nerve, vocal cords, bone, bone marrow, joint tissue, and articular joints.

Examples of the biocompatible scaffold substrates may include, but are not limited to, collagen, gel, TG-gel (e.g., gelatin-transglutaminase crosslinked gel), cross-linked gel, sponge, gelatin, albumin, fibrin, fibrinogen, laminin, fibronectin, vitronectin, a synthetic peptide containing an exposed lysine or glutamine.

A scaffold substrate suitable for use in a composition of the present invention also is not particularly limited so long as it can be enzymatically cross-linked to the growth factor directly and can be selectively digested by an enzyme. Any biocompatible material having surface exposed lysine at a catalytic site recognizable by a transglutaminase (TG) may be used as a scaffold substrate. The scaffold substrate may be in the form of a matrix or a free standing molecule. Exemplary scaffold substrates may include, but are not limited to, TG-gel, gel, cross-linked gel, gelatin, sponge, collagen, albumin, fibrin, fibrinogen, laminin, fibronectin, vitronectin, or a synthetic peptide containing an exposed lysine or glutamine. Those skilled in the art will recognize that depending on the enzyme used, peptides containing certain sequences are more preferable than others. The peptide scaffold substrate is typically derived from a collagen or a derivative thereof, or from a gelatin.

Collagen is known in the art and has been used in different forms for many purposes including the promotion of cell growth and the delivery of pharmaceuticals. Agents for wound healing include bioactive agents, plasticizers, stabilizers, biopolymer, and pharmaceutical combinations.

Cross-linked gels have been used in different forms for many purposes, including the delivery of cells and bioactive agents. An example of this is the polymerization of water soluble macromers containing free radical polymerizable groups such as carbon-carbon double and triple bonds (Hubbell et al., U.S. Pat. No. 5,843,743). These water soluble macromers may form gels by UV or visible light irradiation (Hubbell et al., U.S. Pat. No. 5,801,033). This includes gels which consist of both a core and extensions, where the extensions are designed to reduce tissue, cell and protein adhesions with the gel (Hubbell et al., U.S. Pat. No. 5,626,863).

A process for forming an oriented structure within a biocompatible, bioabsorbable gel is known in the art (Barrows et al., U.S. Pat. No. 5,856,367). A method for forming a gel from serum albumin which reacts with a bifunctional water-soluble cross-linking agent is described in Barrows et al., U.S. Pat. No. 5,583,114.

Methods for making a gel from collagen and a bifunctional polyethylene glycol are known in the art (Rhee et al., U.S. Pat. No. 5,550,187, U.S. Pat. No. 5,523,348, U.S. Pat. No. 5,328,955, U.S. Pat. No. 5,304,595).

Biocompatible matrices or supports based on collagen or mucopolysaccharides are commercially available. For example, a collagen biocompatible matrix which is suitable for use is the Helistat (Integra LifeSciences, Plainsboro, N.J.). A biocompatible matrix produced from hyaluronic acid (Hyaff, Fidia Advanced Biopolymers, Abano Term, Italy) is also suitable for use.

Methods for producing collagen biocompatible matrices are known in the art (U.S. Pat. Nos. 4,193,813, 4,320,201 and 4,970,298) which can in one aspect, be chemically cross-linked, e.g., using a carbodiimide and cross-linking via dehydrothermal treatment. Dehydrothermal treatment is known to make the sponge stiffer and stronger. These processes, as well as cross-linking the matrix with a succinimidyl active ester, and lyophilization, are described in U.S. Pat. No. 4,703,108. A method for forming a lyophilized biopolymer foam, into which collagen is lyophilized, giving a collagen-coated biopolymer foam is known in the art (U.S. Pat. No. 5,948,429). Methods for producing polysaccharide matrices are described by U.S. Pat. No. 5,888,987, which does not involve lyophilization. Other methods are described by U.S. Pat. No. 5,658,582. The density of the matrix must be low enough to allow infiltration of cells and matrix remodeling.

As used herein, the term “demethylation agent or a methylation inhibitor” intends an agent, protein, polypeptide or small molecule drug or composition, that in one aspect, that induces demethylation of 5-methylcytosine by inhibition of DNA methyltransferase, leading to progressive DNA hypomethylation and reactivation of previously silenced genes. In one aspect, the methylation inhibitor comprises, or alternatively consists essentially of, or yet further consists of a cytidine analogue such as 5-azacytidine, 5,6-dihydro-5-azacytidine, 1-β-Darabinofuranosy1-5-azacytidine, 5-aza-2′-deoxycytidine (decitabine) or 1-(β-D-Ribofuranosyl)-2(1H)-pyrimidinone (zebularine), or a non-competitive inhibitor of methyltransferase selected from the group consisting of 4-aminobenzoic acid derivatives such as procainamide and procaine. Methods to make such compounds are known in the art and are described, for example in EP1748792, incorporated herein by reference.

In one specific aspect, the scaffold comprises or alternatively consists essentially of, or yet further consists of, collagen and the cytidine analogue comprises one or more of 5-azacytidine (“Aza” or “Aza-CR”), 5-aza-2′-deoxycytidine (“AZA-CdR”), peudoisocytidine, or 5-fluoro-2′-deoxycytidine.

In one aspect, an activating agent is added to the scaffold to release the growth factor from the matrix. This activating agent may be exogenously added or may be secreted from a target where the growth factor is intended to be delivered, for example, a wound site, or a cell culture. Ideally, the activating agent is an enzyme specific for enzymatically breaking the covalent bond between the growth factor and the peptide carrier. It may enzymatically break down the carrier peptide, but should be harmless for the growth factor. Suitable activating agents may include proteases or metalloproteinases. Exemplary activating agents may include, but not limited to pronase, trypsin, chymopapain, chymotrypsin, papain, collagenase, plasmin, pepsin, elastase, MMP1, MMP2,MMP3, MMP8, MMP9, MMP10, MMP13, MMP14 and MMP18.

Cytokines and other cellular growth factors are known to regulate the growth and function of cells and tissues in general. They are cell messengers and act in low concentrations (nanomolar to femtomolar) by binding to cell receptors, causing a hormone-like action. These molecules are key modulators of cell proliferation, differentiation and matrix production, among other events (Alsberg et al.(2006) Expert Opin Biol Ther. 6, 9, 847-66). Most cytokines and growth factors are expressed under tight control mechanisms. Their gene expression is regulated by environmental stimuli such as infection, cell-cell interactions, extracellular matrix composition and interactions with adhesion molecules or via stimulation with other cytokines However, in some cases, cytokine activity regulation involves the secretion of molecules in a latent form that become “activated” by releasing the cytokine moiety when processes of inflammation, wound healing and tissue repair takes place (Khalil N. (1999) Microbes and Infection, 1, 1255-1263). Many cells produce growth factors in latent form and store them in their extracellular matrix (ECM). Activation can occur at a later time and act on the original cell as an autocrine factor or neighboring cells as a paracrine factor.

Any known biomaterial can be used in the present disclosure, non-limiting examples of such include natural or non natural materials, modified or derivatized natural polymers, modified or derivatized non-natural polymers, and combinations Synthetic polymers and copolymers, such as (e.g. poly-L-lactic acid (PLLA), poly(lactic-co-glycolic acid (PLGA), polyethylene glycol (PEG), polyethylene-co-vinylacetate, poly caprol lactone, poly hydroxyl alkanoates, polyesters and others), natural polymers (e.g. polysaccharides, proteins, proteoglycans, lipids, all types of collagen, hyaluronic acid, starch, chitosan, chitin, dextran, pullulan, agarose, methycecllulose, alginate, and others known in the art) or combinations thereof, either degradable or non-degradable, crosslinked or non-crosslinked, but not limited to these. The biomaterial can be an inorganic material such as calcium phosphate and is selected from amorphous calcium phosphate, poorly crystalline hydroxyapatite, nanocrystalline hydroxyapatite, stoichiometric hydroxyapatite, calcium deficient hydroxyapatite, substituted hydroxyapatites, tri calcium phosphate, tetracalcium phosphate, dicalcium phosphate dihydrate, and monocalcium phosphate. ceramic materials such as hydroxyapatite, soluble glasses and ceramic forms, metallic materials or composite materials, and combinations thereof, including combinations with previous described possibilities.

The biomaterial can be in the form of a gel, sol-gel, hydrogel, membrane, fibrous structures, nano or microfibers, micro or nanowires, porous sponges, woven or non-woven meshes, other known forms, or any combination thereof. The biomaterial can be prepared using different procedures such as gas foaming/particulate, freeze-drying, electrospinning, thermal induced phase separation, injectable scaffolds, but not limited to these.

Cytokines and other cellular growth factors are known to regulate the growth and function of cells and tissues in general. They are cell messengers and act in low concentrations (nanomolar to femtomolar) by binding to cell receptors, causing a hormone-like action. These molecules are key modulators of cell proliferation, differentiation and matrix production, among other events (Alsberg et al. (2006) Expert Opin Biol Ther. 6, 9, 847-66). Most cytokines and growth factors are expressed under tight control mechanisms. Their gene expression is regulated by environmental stimuli such as infection, cell-cell interactions, extracellular matrix composition and interactions with adhesion molecules or via stimulation with other cytokines However, in some cases, cytokine activity regulation involves the secretion of molecules in a latent form that become “activated” by releasing the cytokine moiety when processes of inflammation, wound healing and tissue repair takes place (Khalil N. (1999) Microbes and Infection, 1:1255-1263). Many cells produce growth factors in latent form and store them in their extracellular matrix (ECM). Activation can occur at a later time and act on the original cell as an autocrine factor or neighboring cells as a paracrine factor.

Any growth factor provided can be used in the present disclosure. The growth factor can be any molecule capable of stimulating cell factor, migration, dedifferentiation, redifferentiation or differentiation. For example, the growth factor may be, but is not limited to, TGFI3, epidermal growth factor (EGF), platelet derived growth factor (PDGF), nerve growth factor (NGF), colony stimulating factor (CSF), hepatocyte growth factor, insulin-like growth factor, placenta growth factor); differentiation factor; a cytokine e.g. interleukin, (e.g. IL-1, IL-2, IL-3, IL-4, IL-5, IL-6, IL-7, IL-8, IL-9, IL-10, IL-I 1, IL-12, IL-13, IL-14, IL-15, IL-16, IL-17, IL-18, IL-19, IL-20 or IL-21, each of either α or β interferon (e.g. IFN-α, IFN-β and IFN-γ), tumor necrosis growth factor (TNF), IFN-γ inducing growth factor (IGIF), bone morphogenetic protein (BMP); a chemokine (e.g. MIPs (Macrophage Inflammatory Proteins) e.g. MIP Iα and MIP1β; MCPs (Monocyte Chemotactic Proteins) e.g. MCP1, 2 or 3; RANTES (regulated upon activation normal T-cell expressed and secreted)) and trophic factors. For example, the growth factor may be selected from the group of TGF-β1, TGF-β2, TGF-β3, TGF-β4, β5, or any other member of the TGF-β superfamily including activins, inhibins and bone morphogenetic proteins including BMP1, BMP2, BMP3, BMP4, BMP5, BMP6, or BMP7. Preferably, the bioactive molecule is derived from the species to be treated e.g. human origin for the treatment of humans.

In one aspect, the scaffold comprises, or yet consists essentially of, or yet further consists of collagen. In another aspect the scaffold comprises, or yet consists essentially of, or yet further consists of a cross-linked matrix such as TG-Gel, also referred to herein as Col-Tgel.

In a further aspect, the scaffold comprises collagen and the demethylation agent or a methylation inhibitor comprises one or more of Aza-CR or Aza-CDR. In another aspect, the scaffold comprises TG-Gel and the demethylation agent or a methylation inhibitor comprises one or more of Aza-CR or Aza-CDR. In another aspect, the scaffold comprises TG-Gel and the demethylation agent or a methylation inhibitor comprises one or more of Aza-CR or Aza-CDR.

In a further aspect, the scaffold comprises collagen and the demethylation agent or a methylation inhibitor comprises one or more of Aza-CR or Aza-CDR and a target specific growth factor, e.g. BMP such as BMP2. In another aspect, the scaffold comprises TG-Gel and the demethylation agent or a methylation inhibitor comprises one or more of Aza-CR or AzaCDR and a target specific growth factor, e.g. BMP such as BMP2. In another aspect, the scaffold comprises TG-Gel and the demethylation agent or a methylation inhibitor comprises one or more of Aza or Aza-CR and a target specific growth factor, e.g. BMP such as BMP2. These scaffolds are useful to promote one or more of osteogenesis, myogenesis, cardiocmyocyte generation, and adipogenesis.

Applicant has unexpectedly discovered that very low doses of the demethylation agent or a methylation inhibitor, in the absence of exogenously added cells or tissue, will promote tissue growth or regeneration. Such amounts include from about 1 ng per cm³ to about 500 ng per cm³ of scaffold substrate, or alternatively from about 2 ng per cm³ to about 250 ng per cm³ of scaffold substrate, or alternatively from about 5 ng per cm³ to about 200 ng per cm³ of scaffold substrate, or alternatively from about 10 ng per cm³ to about 150 ng per cm³ of scaffold substrate or from about 12 ng to about 120 ng per cm³ of scaffold substrate.

In one aspect, the scaffold as described above, comprises, or alternatively consists essentially of, or yet further consists of, an effective amount of a target specific growth factor. As used herein, the term “target specific growth factor” designates a small molecule, protein or polypeptide, gene, growth factors, cytokine and hormone; miRNA and exosome, that is known to help cells immature cells differentiate and preserve the biological functions of the repaired tissues. For example, in bone repair and regeneration, bone morphogenetic proteins (BMPs) are the growth factors that promote and regulate bone formation. In particular, BMP-2 has the ability to regulate the differentiation of osteoblastic progenitor cells and the ability to transdifferentiate non-osteogenic cells towards an osteoblastic lineage in vitro. By recruiting progenitor cells, BMP-2 is capable of inducing new bone formation at ectopic and orthotropic sites. In clinical studies, BMP-2 has the potential to replace autografts by inducing new bone growth for spinal fusion and non-union bone healing.

Non-limiting examples of bone morphogenetic proteins (BMPs) include without limitation BMP10, BMP15, BMP2, BMP3, BMP4, BMP5, BMP6, BMP7, BMP8A, BMP8B. Other target specific growth factors include without limitation several TGF-beta (“TGB”) factors, e.g. TGFB1, TGFB2, TGFB3. Other target specific growth factors include , GDF1, GDF10, GDF11, GDF15, GDF2, GDF3, GDF3A, GDFS, GDF6, GDF7, GDF8, GDF9, GDNF, INHA, INHBA, INHBB, INHBC, INHBE, LEFTY1, LEFTY2, MSTN, NODAL, NRTN, PSPN, AMH, ARTN, FGF, PDGF, VEGF, IGF, insulin and NGF.

In one aspect, the target specific growth factor is present in the scaffold in a specific concentration, e.g., from about 10 ng per cm³ to about 1000 ng per cm³ of scaffold substrate, or alternatively from about 25 ng per cm³ to about 750 ng per cm³ of scaffold substrate, or alternatively from about 50 ng per cm³ to about 500 ng per cm³ of scaffold substrate, or alternatively from about 50 ng per cm³ to about 350 ng per cm³ of scaffold substrate.

In one aspect, the scaffold as described above comprises more than one growth factor, e.g., TGFB and IGF or TGFB and GDF7.

In a further aspect, the scaffolds of this disclosure are further combined with a pharmaceutically acceptable carrier. This can be done in order to prepare a pharmaceutical composition.

In one aspect, the scaffold, alone or in combination with the carrier, are lyophilized or processed for ease of transport and use. Kits containing same are further provided, with the materials and instructions necessary for use. The kits comprise a scaffold or pharmaceutical composition according to the present invention packaged with directions for its use.

The scaffolds of this disclosure can include other components that will facilitate clinical use of the scaffold without materially affecting biological function, such as a pharmaceutically acceptable carrier. Non-limiting examples of such are described above.

### Loading Demethylation Drug to the Matrix

5-azacytidine (also known as azacitidine (Aza) and 4-amino-1-(3-D-ribofuranosyl-1,3,5-triazin-2(1H)-one; Nation Service Center designation NSC-102816; CAS Registry Number 32067-2) has undergone NCI-sponsored clinical trials for the treatment of myelodysplastic syndromes (MDS). See Kornblith et al., J. Clin. Oncol. 20(10): 2441-2452 (2002) and Silverman et al., J. Clin. Oncol. 20(10): 2429-2440 (2002). 5-azacytidine may be defined as having a formula of C₈H₁₂N₄O₅, a molecular weight of 244.20 and a structure as shown below of Formula (I):

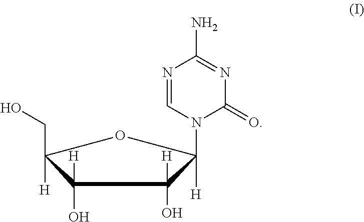

Any inhibitor such as Aza can be loaded to the biomaterials in the form of powders, suspensions, emulsions, solutions, syrups. Suitable buffers encompass, but are not limited to, phosphate, citrate, tartrate, succinate, and the like. The scaffolds of the disclosure can be formulated so as to provide quick, sustained, controlled, or delayed release of the drug substance. The inhibitor, e.g., Aza, can be dispersed onto a biomaterial, encapsulated into the material, or through linker to bind to material in which sustained release of the pharmaceutical agents aza for at least 5 hour and preferably for more than 1, 3, or even 5 days. The rate of release from the material may be varied as described herein, i.e. by regulating the rate of dissolution, the rate of permeability, or the swelling rates, which in turn may be controlled by controlling the pH, moisture and temperature of the environment, and chemical properties of the polymeric matrix, such as for example its size, shape and thickness. The inhibitor, e.g., Aza, can be mixed, dispersed or bonded to another chemical moiety that may reduce its solubility.

### In Vivo Tissue Repair and Regeneration

The disclosed scaffolds are useful in method for treating a subject, comprising, or alternatively consisting essentially of, or yet further consisting of, implanting into the subject a scaffold as described above, thereby treating the subject. In one aspect, “treatment” intends tissue repair or regeneration by local administration by implanting into the subject at the location to be treated, a scaffold as described above. Thus, this disclosure also provides a method for promoting tissue regeneration in a subject comprising implanting into the subject a scaffold as described above. In one aspect the scaffold material comprises collagen and/or TG-Gel and the target specific growth factor comprises BMP-2. This composition is useful in repairing or regenerating the bone or tissue of the cranium or the spine.

In another aspect the scaffold material comprises collagen and/or TG-Gel and the target specific growth factor comprises GDF11. This composition is useful in repairing or regenerating muscle tissue.

In another aspect, the scaffold material comprises, or alternatively consisting essentially of, or yet further consists of collagen and/or TG-Gel and the target specific growth factor comprises TGFB and IGF and is useful in a method for promoting tendon repair.

In another aspect, the scaffold material comprises, or alternatively consisting essentially of, or yet further consists of collagen and the target specific growth factor comprises insulin and is useful in a method for regenerating adipose tissue when implanted in a subject.

In another aspect, the scaffold material comprises, or alternatively consisting essentially of, or yet further consists of collagen and the target specific growth factor comprises NGF and is useful in a method for regenerating nervous tissue when implanted in a subject.

### Materials and Methods

### TG-Gel

A gelatin solution designated as “TG-Gel” of 3, 4.5, 6, 7.7 or 9% was made as described below.

Gelatin (Type A 225 bloom, Sigma Aldrich) was dissolved and autoclaved in distilled water to make a 10% gelatin stock. The autoclaved gelatin was aliquoted and stored at 4° C. until use. A 2% percent gelatin solution was made by diluting from a 10% gelatin stock at 37° C. with BMP-2 buffer (25 mM tricine, pH 7.2, 15 mM sucrose, 1.7 mM NaCl, and 0.01% Tween 80).

Microbial transglutaminase (ACTIVA TI Ajinomoto, Japan, TGase) from Streptomyces mobaraense was purified using a Sepharose Fast Flow column. Briefly, 3 g of crude TGase were dissolved in a phosphate buffer (20 mM phosphate and 2 mM EDTA, pH 6.0) and gently mixed with 3 ml of pre-equilibrated S Sepharose Fast Flow beads (Sigma). After incubation at 4° C. overnight with occasional vortexing, the protein solution and beads mixture were batch loaded into a column. After washing with 4 volumes of phosphate buffer, TGase was eluted with eluting buffer (phosphate buffer with 800 mM NaCl). Protein concentration was monitored by the Bradford method (Bio-Rad) utilizing BSA as a standard. BMP-2 (R&D systems) was kept in stock concentrations of 20 ng/μL at −20° C. in buffer solution (5 mM glutamic acid, 2.5% glycine, 0.5% sucrose, and 0.01% Tween 80).

### Low Dose of 5-Azacytidine (“Aza”) Activates Cell Proliferation in 3D Culture

10⁶/m1 rat adipose derived cells was suspended in the gelatin-transglutaminase crosslinked gel also referred to TG-Gel. Gel concentrations ranged from 3%, 4.5%, 6%, and 7.5%. A cell suspension in 20 μL volume was transferred in each well of 48 well culture plate followed by incubation at 37° C. for 30 min for gelation. 1.0 ml of medium (10%FBS/DMEM) with 5 nM Aza was added in each well and the plate was placed in a 5% CO₂ 37° C. incubator for 5 day culture. At day 5, cell proliferation marker (Ki-67) immune histochemical staining was performed on 3D culture. Medium without Aza was used as control.

The detection of Ki67, a proliferation marker, was performed using Anti-Ki-67 (Rabbit Monoclonal antibody, Clone SP6, from Thermo Scientific). The results are shown in Table 1.

TABLE 1
Anti - Ki67

| | w/o AZA-CR | | w/AZA-CR | |
|---|---|---|---|---|
| TG-Gel | Center | Peripheral | Center | Peripheral |
| 3% TG-Gel | — | + | — | + |
| 4.5% TG-Gel | ~30% | ++++ | ~30% | ++++ |

| | | | |
|---|---|---|---|
| 6% TG-Gel | — | + | Evenly distributed (~80%) |
| 7.5% TG-Gel | — | + | Evenly distributed (~50%) |

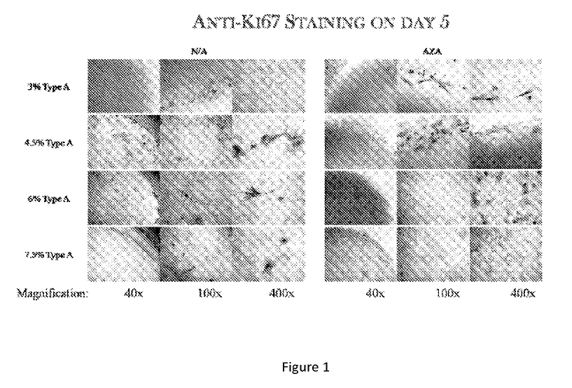

For the 3%-4.5% gel condition, there is no difference in Ki67 positive staining in control and Aza group. For the 6%-7% gel condition, significantly more positive stained cells are in Aza group, and they are evenly distributed the whole 3D construct. The results are shown in FIG. 1.

This example shows that low doses of Aza can activate quiescent cells and that a single dose is sufficient.

### Aza Activated Cells Are Sensitive to Microenvironmental Signals

### Osteogenic Differentiation in in Vitro 3D Conditions

Using the matrix as described above, the medium was changed to regular growth medium at day 5 and maintained for 14 days. Alkaline phosphatase (ALP) activity and staining were performed to characterize early osteogenesis at day 5 and calcium staining at day 14. ALP activity was determined by biochemical assay based on conversion of p-nitrophenyl phosphate to p-nitrophenol (pNPP), which was measured spectrophotometrically at 405 nm absorbance. Briefly, TG-Gel-Cell, a transglutaminase crosslinked gelatin gel and cell mixture, construct was washed twice with PBS and homogenized with buffer solution (0.25% TritonX-100 in PBS). After three freezing-thawing cycles, cells were released from TG-Gel by adding 50 μL of 0.25% trypsin and incubated at 37° C. for 4 hours. ALP activity was then quantified by using p-NPP as a substrate (Pierce, USA). The absorbance was measured by a multiplate reader (Molecular Devices, CA) after 30 minutes of incubation at 37° C. The ALP activity was specified as an arbitrary unit of ALP absorbance that normalized with cell numbers. Alizarin Red S staining as late marker of osteogenic differentiation was performed to determine the degree of calcium deposition as described. Briefly, TG-Gel-Cell construct was washed twice with distilled water (dH2O), fixed in 10% neutral formalin for 10 min, and stained with 40 mM alizarin red S (pH 4.2, Sigma, Mo.) for 30 min. Prior to photography, the stained sample was washed twice with dH2O to remove nonspecific precipitation. Positive red-orange staining represented calcium deposited by the differentiated cells. Images were captured and presented for analysis of late-stage osteogenic differentiation. After photography, dH2O was removed and sample was incubated in 500 ul of 10% (w/v) cetylpyridinium chloride for 10 min to release bounded calcium. The supernatant was collected and absorbance was measured by a multiplate reader (Molecular Devices, CA).

Alizarin Red S staining as late marker of osteogenic differentiation was performed to determine the degree of calcium deposition.

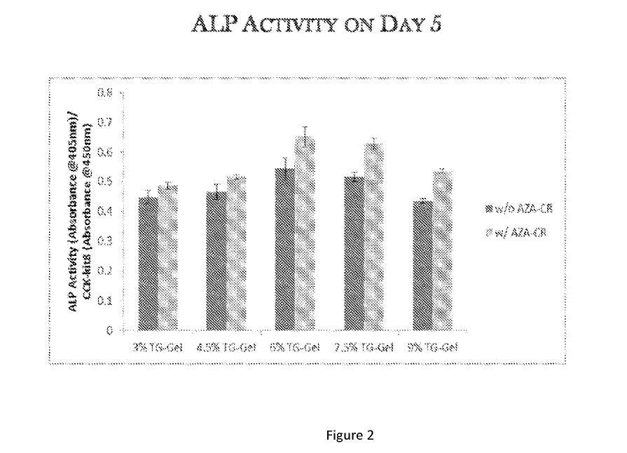

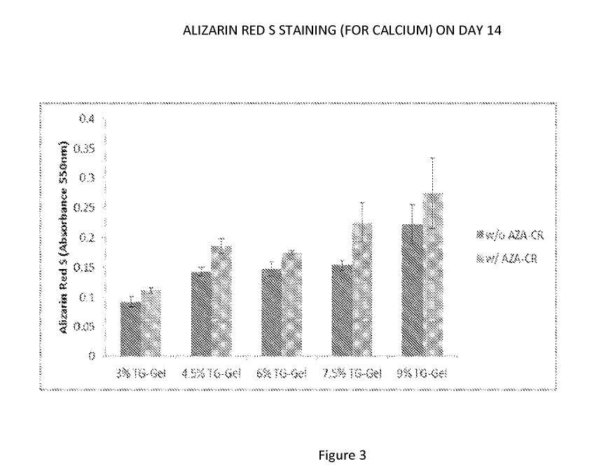

AZA-CR enhanced ALP activity and calcium deposition. The results are shown in FIG. 2 for ALP activity and in FIG. 3 for calcium deposition. ALP peaked at 6% TG-Gel (day 5) and calcium deposition peaked at 9% TG-Gel (day 14) with and without AZA-CR. The trend is similar to the osteogenesis trend of myoblast C2C12 in medium with and without BMP-2.

### Myogenesis Differentiation

A myogenic marker such as MyoD was stained on day 14 adipose cell 3D culture. The anti-MyoD antibody is a monoclonal available from Pierce. The results are shown in FIG. 4 and in Table 2.

TABLE 2
Anti-MyoD on day 14

| | w/o AZA-CR | | w/AZA-CR | |
|---|---|---|---|---|
| TG-Gel | Center | Peripheral | Center | Peripheral |
| 3% TG-Gel | − | + | + | +/++ |
| 4.5% TG-Gel | − | +++ | ~80% Evenly Distributed | |
| 6% TG-Gel | − | + | ~70% Evenly Distributed | |
| 7.5% TG-Gel | − | + | − | ++/+++ |
| 9% TG-Gel | − | + | − | ++ |

### Cardiomyocyte Differentiation

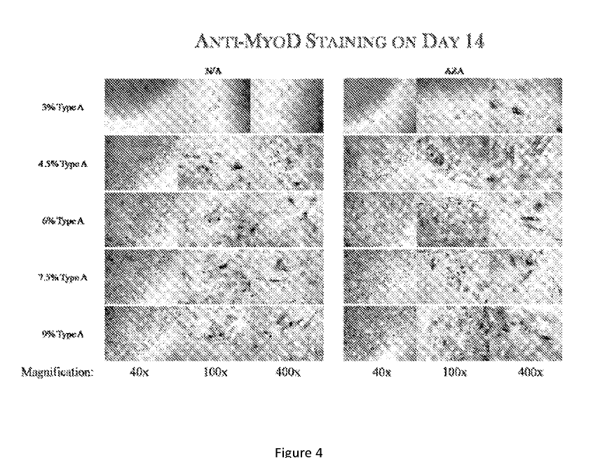

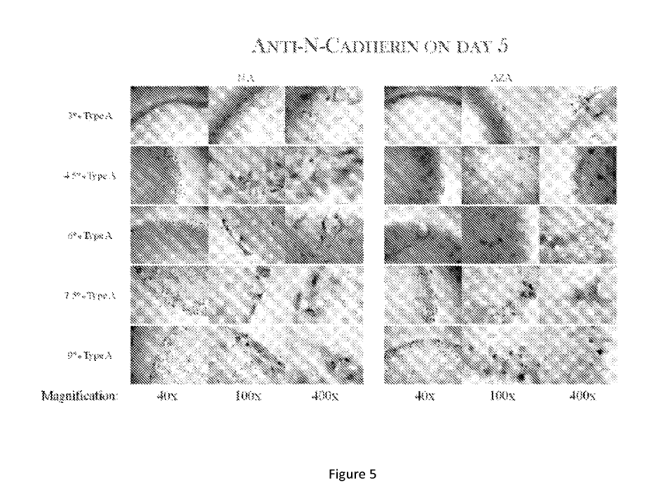

N-cadherin positive cells (from MSC or adipose cells) showed high ability for cardiomyocyte differentiation. The results are shown in FIG. 5 and in Table 3.

TABLE 3
Anti-N-Cadherin

| | |
|---|---|
| w/o AZA-CR | w/AZA-CR |

| TG-Gel | Center | Peripheral | Center | Peripheral |
|---|---|---|---|---|
| 3% TG-Gel | — | + | — | + |
| 4.5% TG-Gel | <10% | ++++ | ~50% | +++++ |
| 6% TG-Gel | — | + | — | +/++ |

Enhancement of Adipogenesis

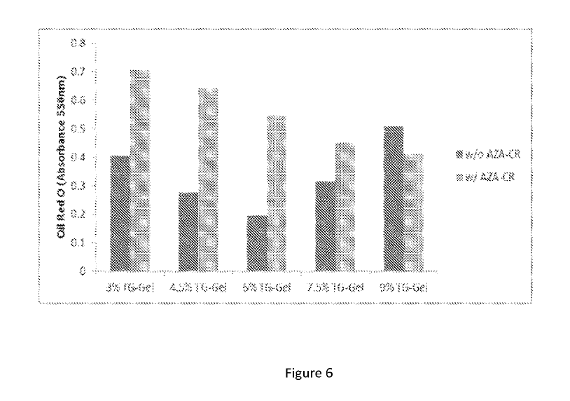

AZA-CR demonstrated enhancement of adipogenesis with a peak at 3% TG gel. The results are shown FIG. 6. Oil Red O, which stains lipids, is used to assay adipogenesis.

### In Vivo Studies

### Cartilage Defect Model

Fisher rats (7-8-weeks old, 200-250 g each) were used in this study. They were randomly enrolled into two experimental groups,A, 4.5% Tg-Gel +50 μL PBS and B, 4.5% Tg-Gel +50 μL Aza (500 nM), 4 animals per group. A 3-mm diameter×2-mm deep osteochondral defect was drilled into the rat trochlear groove, using a dermal biopsy punch (Miltex, Inc.). Implant gel was placed in the defect area. Skin was closed with 4-0 suture. At 12 days postsurgery, animals were euthanized by CO₂ inhalation and knee joint was retrieved for histology.

At 7 days, in gel only group, fibrous tissue filled the defect site. In the gel matrix with Aza groups, area adjacent to subchondral bone showed neo cell infiltration. Cell morphology resembles chondrogenic cells. The results are shown in FIG. 7 (4.5% Tg-Gel, control, 7 days, left panel; 4.5% T-Gel +250 nmol Aza, 7 days, right panel).

### Aza-Collagen-BMP2 Matrix Induces Ectopic Bone Formation in Young Animals

Collagen sponges (derived from porcine tendon collagen) were prepared. Sponges were cut into 5-mm diameter disks, 2-mm in height, for implantation. Collagen sponge with 50 μL of 50 nM, 250 nM or 500 nM Aza-CR was prepared. PBS was used as a control. Before implantation, 0.1 μg of rhBMP-2 was added to each disk and implanted intramuscularly in the abdominal muscle pouch of 8 week old male Fisher-344 rats. After 4 weeks of implantation, explants were cut into two-halves. One half was evaluated histologically with hematoxylin and eosin (H&E) for visualization of cells and tissue formation. Alkaline phosphatase was determined in the same half of the homogenized tissue.

The results are shown in FIG. 8 (8A: collagen; 8B: Collagen+100 ng; 8C: Collagen+100 ng+50 nM Aza-CR; 8D: Collagen+100 ng+500 nM Aza-CR) and in FIG. 9 for alkaline phosphatase.

### Aza-Collagen-BMP-2 Matrix Induces Ectopic Bone Formation in Aqed Animals

Collagen sponges (derived from porcine tendon collagen) were prepared. Sponges were cut into 5-mm diameter disks, 2-mm in height, for implantation. Collagen sponge was loaded with 250 nM, 500 nM Aza-CR or Aza-CDR respectively. Before implantation, 0.1 μg of rhBMP-2 was added to each disk and implanted intramuscularly in the abdominal muscle pouch of >8 month old male SD rats. After 4 weeks of implantation, explants were evaluated histologically with hematoxylin and eosin (H&E) for visualization of cells and tissue formation.

The results are shown in FIG. 10 and in Table 4.

TABLE 4

| Samples description | Bone score |
|---|---|
| Collagen + PBS | 0 |
| Collagen + 100 ng BMP-2 | 0 |
| Collagen + 100 ng BMP-2 + CDR 250 nM | 0 |
| Collagen + 100 ng BMP-2 + CDR 500 nM | 0 |
| Collagen + 100 ng BMP-2 + CR 250 nM | 4 |
| Collagen + 100 ng BMP-2 + CR 500 nM | 4 |

Bone Formation at the Bony Defect Site

Cranial Defect Model

Collagen matrix (as described above) was prepared and cut into 5 mm discs. An 5 mm diameter circular opening was created in the skull of >8 month old retired rats using circular drill bit including a lip to restrict the depth of the circular cutting. 500 nM of Aza was applied directly into the collagen sponge before implantation. Control included collagen discs without added Aza-CR but same volume of PBS. After four weeks of implantation, bone growth and healing status were evaluated histologically. The results are shown in FIG. 11.

At the bony site, without adding exogenous growth factor BMP-2 still induces significant bone remodeling.

### Spinal Fusion (Matrix+Aza Without Growth Factors)

Fisher 344 rats ranging from 8 to 10 weeks of age were used. Test groups included: collagen sponge+Aza-CR, 500 nM or Collagen+PBS as control. A 3 mm×4 mm×20 mm or 240 mm³ volume sponge holds 100 μL of solution. After anesthesia induction by ketamine and xylazine a posterior midline incision was made over the skin of the lumbar spine. Two separate paramedian incisions were made in the lumbar fascia 3 mm from the midline. The transverse processes of the L4 and L5 vertebrae were exposed by separating the back muscles and decorticated with an electric-driven burr (Stryker) until a blush of cancellous bone was observed.

Two implants, each 0.3 mL in volume, were placed over the decorticated transverse processes on each side. Fascia and skin incisions were closed with 4-0 Vicryl absorbable sutures. After surgery, animals were housed in separate cages and allowed to eat and drink ad libitum. About 0.05 mg/kg buprenorphine was administered twice daily for 2 days postoperatively for analgesia. All animals were sacrificed at 4 weeks.

Manual Palpation: Spines were dissected and harvested after euthanasia. Three blinded observers scored spines either fused or not fused by applying flexion and extension forces to the intervertebral space. Only specimens with no mobility against the applied forces at the L4/L5 region were judged as a successful fusion. Any motion detected at either side between the transverse processes was considered a failure of fusion. At least two observers needed to have scored a spine fused in order for it to be deemed fused.

Radiographic Assessment: Posteroanterior radiographs were performed 4 weeks after implantation using a Faxitron.

Histology: Histology was performed on explants.

The results of manual palpation are shown in Table 5. The results with respect to radiographic assessment and histology are shown in FIG. 12.

TABLE 5

| Implant | Collagen + PBS | Collagen + 500 nM Aza |
|---|---|---|
| Palpation score (0-4) | 0 | 2-3 |

### Muscle Regeneration

Volumetric Muscle Loss (VML) was created in the rat. Using aseptic technique, a longitudinal incision was made along the lateral aspect of the lower leg using a scalpel. The skin was separated from the fascia by blunt dissection. With the TA muscle exposed, a flat spatula was then inserted under the TA muscle (but over the EDL), which flattened the TA muscle in preparation for the creation of the surgical defect. The surgical defect was created by 5 mm skin puncher. Defects were filled with 100 IA of Col-Tgel (transglutaminase crosslinked gelatin gel) with or without Aza-CR. Skin was closed with 4-0 suture. Animals were sacrificed at 1 week and 4 weeks for histology analysis using H&E for morphology and stem cell marker OCT-4 IHC for an immunological specific indicator of stem cell growth. The results are shown in FIG. 13; the top panels show the results of H&E staining for morphology and the bottom panels show the results of OCT-4 IHC staining for immunological specific indication of stem cell growth; the left panels are without Aza-CR and the right panels are with Aza-CR.

Retired Breeder male Sprague-Dawley rats (>6 month old, 400-450 grams) underwent a bilateral supraspinatus detachment and repair surgery. Shoulders were randomly assigned to have no scaffold (control), a TG-Gel scaffold (gel), or a gel+Aza (500 nM) (gel Aza) to augment the repair. Detached tendon was suture back by modified Mason-Allen stitch. Scaffolds were injected at the suture site by overlay along the anterior and posterior borders of supraspinatus tendon. Rats were allowed normal cage activity and were euthanized at 4 weeks after injury and repair.

### Cartilage Defect Model

Fisher rats (7-8-weeks old, 200-250 g each) were used in this study. They were randomly enrolled into two experimental groups,A, 4.5% Tg-Gel+50 μL PBS and B, 4.5% Tg-Gel+50 μL Aza (500 nM), 4 animals per group. A 3-mm diamete×2-mm deep osteochondral defect was drilled into the rat trochlear groove, using a dermal biopsy punch (Miltex, Inc.). Implant gel was placed in the defect area. Skin was closed with 4-0 suture. At 12 days postsurgery, animals were euthanized by CO₂ inhalation and knee joint was retrieved for histology.

The results are shown in FIG. 14. In FIG. 14, the left panel shows the 4.5% Tg-gel control at 7 days and the right panel the 4.5% Tg-gel+250 nmole Aza at 7 days.

### ADVANTAGES OF THE INVENTION

The present invention provides compositions and methods that provide a biocompatible scaffold that has versatile uses in tissue repair and regeneration of a large number of tissues, including bone, cartilage, muscle, tendon, nervous tissue, and adipose tissue. The compositions and methods according to the present invention promote differentiation so that tissues that are biologically functional and compatible with surrounding tissues are formed. The biocompatible scaffolds of the present invention are well tolerated and can be used together with other agents or methods for tissue repair or regeneration.

Compositions according to the present invention possess industrial applicability as compositions of matter that have uses in medicine. Methods according to the present invention possess industrial applicability as methods for the preparation of a medicament for tissue repair and regeneration.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

What is claimed is:

1. A biocompatible scaffold, comprising a biocompatible scaffold substrate and an amount of a demethylation agent or a methylation inhibitor effective to induce and/or promote tissue repair and regeneration.

2. The scaffold of claim 1, wherein the biocompatible scaffold comprises one or more of a material that is a natural or non-natural material selected from the group consisting of:
(i) modified or derivatized natural polymers;
(ii) modified or derivatized non-natural polymers;
(iii) combinations of modified or derivatized natural polymers and modified or derivatized non-natural polymers;
(iv) collagen;
(v) gelatin;
(vi) albumin;
(vii) fibrin;
(viii) fibrinogen;
(ix) laminin;
(x) fibronectin;
(xi) vitronectin;
(xii) a gel;
(xiii) a sponge;
(xiv) TG-gel;
(xv) a synthetic peptide containing an exposed lysine or glutamine;
(xvi) a synthetic polymer or copolymer selected from the group consisting of poly-L-lactic acid (PLLA), poly(lactic-co-glycolic acid (PLGA), polyethylene glycol (PEG), polyethylene-co-vinylacetate, poly caprol lactone, poly hydroxyl alkanoates, and polyesters;
(xvii) hyaluronic acid;
(xviii) polysaccharides;
(xix) proteins;
(xx) proteoglycans;
(xxi) lipids;
(xxii) starch;
(xxiii) chitosan;
(xxiv) dextran;
(xxv) pullulan;
(xxvi) agarose;
(xxvii) methylcellulose;
(xxviii) alginate;
(xxix) a modified or derivatized polymer derived from (iii) to (xxviii);
(xxx) calcium phosphate, wherein the form of calcium phosphate is selected from the group consisting of amorphous calcium phosphate, poorly crystalline hydroxyapatite, nanocrystalline hydroxyapatite, stoichiometric hydroxyapatite, calcium deficient hydroxyapatite, substituted hydroxyapatites, tri calcium phosphate, tetracalcium phosphate, dicalcium phosphate dihydrate, and monocalcium phosphate;
(xxxi) ceramic materials;
(xxxii) soluble glasses;
(xxxiii) metallic materials;
(xxxiv)composite materials; and
(xxxv) combinations thereof;
wherein the material is degradable or non-degradable and is crosslinked or non-crosslinked.

3. The scaffold of claim 1, wherein the methylation inhibitor comprises a cytidine analogue.

4. The scaffold of claim 3, wherein the cytidine analogue induces demethylation of DNA containing 5-methylcytosine by inhibition of cytosine methylation by inhibition of DNA methyltransferase.

5. The scaffold of claim 4, wherein the cytidine analogue comprises at least one of 5-azacytidine, 5-aza-2′-deoxycytidine, peudoisocytidine, and 5-fluoro-2′-deoxycytidine.

6. The scaffold of claim 1, wherein the amount of the demethylation agent or the methylation inhibitor is from about 1 ng per cm³ to about 500 ng per cm³ of scaffold substrate.

7. The scaffold of claim 6, wherein the amount of the demethylation agent or the methylation inhibitor is from about 5 ng per cm³ to about 250 ng per cm³ of scaffold substrate.

8. The scaffold of claim 7, wherein the amount of the demethylation agent or the methylation inhibitor is from about 5 ng per cm³ to about 150 ng per cm³ of scaffold substrate.

9. The scaffold of claim 1 further comprising an effective amount of one or more of a target specific growth factor, a cytokine, an activating agent or a signaling molecule.

10. The scaffold of claim 9, wherein the scaffold further comprises an effective amount of a target specific growth factor, and wherein the target specific growth factor is selected from the group consisting of insulin, BMP10, BMP15, BMP2, BMP3, BMP4, BMPS, BMP6, BMP7, BMP8A, BMP8B, TGFB, TGFB1, TGFB2, TGFB3, GDF1, GDF10, GDF11, GDF15, GDF2, GDF3, GDF3A, GDFS, GDF6, GDF7, GDF8, GDF9, GDNF, INHA, INHBA, INHBB, INHBC, INHBE, LEFTY1, LEFTY2, MSTN, NODAL, NRTN, PSPN, AMH, ARTN, FGF, PDGF, VEGF, IGF, and NGF.

11. The scaffold of claim 10, wherein the effective amount of the target specific growth factor is from about 10 ng per cm³ to about 1000 ng per cm³ of scaffold substrate.

12. The scaffold of claim 11, wherein the effective amount of the target specific growth factor is from about 25 ng per cm³ to about 750 ng per cm³ of scaffold substrate.

13. The scaffold of claim 12, wherein the effective amount of the target specific growth factor is from about 50 ng per cm³ to about 500 ng per cm³ of scaffold substrate.

14. The scaffold of claim 13, wherein the effective amount of the target specific growth factor is from about 50 ng per cm³ to about 350 ng per cm³ of scaffold substrate.

15. The scaffold of claim 10, wherein the target specific growth factor comprises TGFB and IGF.

16. The scaffold of claim 10, wherein the target specific growth factor comprises TGFB and GDF7.

17. A biocompatible scaffold, consisting essentially of a biocompatible scaffold substrate and an amount of a demethylation agent or a methylation inhibitor, effective to induce and/or promote tissue repair and regeneration.

18. The scaffold of claim 17, wherein the biocompatible scaffold consists essentially of one or more of collagen, gelatin, polyglycolic and polylactic acids, fibrin, or a fibrin glue mixture.

19. The scaffold of claim 17, wherein the methylation inhibitor consists essentially of a cytidine analogue.

20. The scaffold of claim 19, wherein the cytidine analogue induces demethylation of DNA containing 5-methylcytosine by inhibition of cytosine methylation by inhibition of DNA methyltransferase.

21. The scaffold of claim 20, wherein the cytidine analog consists essentially of at least one of 5-azacytidine, 5-aza-2′-deoxycytidine, peudoisocytidine, and 5-fluoro-2′-deoxycytidine.

22. The scaffold of claim 17, wherein the amount of the demethylation agent or the methylation inhibitor is from about 1 ng per cm³ to about 500 ng per cm³ of scaffold substrate.

23. The scaffold of claim 22, wherein the amount of the demethylation agent or the methylation inhibitor is from about 5 ng per cm³ to about 250 ng per cm³ of scaffold substrate.

24. The scaffold of claim 23, wherein the amount of the demethylation agent or the methylation inhibitor is from about 5 ng per cm³ to about 150 ng per cm³ of scaffold substrate.

25. The scaffold of claim 17 further consisting essentially of an effective amount of one or more of a target specific growth factor, a cytokine, an activating agent or a signaling molecule.

26. The scaffold of claim 25, wherein the scaffold further consists essentially of an effective amount of a target specific growth factor, and wherein the target specific growth factor is selected from the group consisting of insulin, BMP10, BMP15, BMP2, BMP3, BMP4, BMP5, BMP6, BMP7, BMP8A, BMP8B, TGFB, TGFB1, TGFB2, TGFB3, GDF1, GDF10, GDF11, GDF15, GDF2, GDF3, GDF3A, GDF5, GDF6, GDF7, GDF8, GDF9, GDNF, INHA, INHBA, INHBB, INHBC, INHBE, LEFTY1, LEFTY2, MSTN, NODAL, NRTN, PSPN, AMH, ARTN, FGF, PDGF, VEGF, IGF, and NGF.

27. The scaffold of claim 26, wherein the effective amount of the target specific growth factor is from about 10 ng per cm³ to about 1000 ng per cm³ of scaffold substrate.

28. The scaffold of claim 27, wherein the effective amount of the target specific growth factor is from about 25 ng per cm³ to about 750 ng per cm³ of scaffold substrate.

29. The scaffold of claim 28, wherein the effective amount of the target specific growth factor is from about 50 ng per cm³ to about 500 ng per cm³ of scaffold substrate.

30. The scaffold of claim 29, wherein the effective amount of the target specific growth factor is from about 50 ng per cm³ to about 350 ng per cm³ of scaffold substrate.

31. The scaffold of claim 26, wherein the target specific growth factor consists essentially of TGFB and IGF.

32. The scaffold of claim 26, wherein the target specific growth factor consists essentially of TGFB and GDF7.

33. A method for treating a subject, comprising implanting into the subject an effective amount of the scaffold of claim 1 into the subject, thereby treating the subject.

34. A method for treating a subject, comprising implanting into the subject an effective amount of the scaffold of claim 17 into the subject, thereby treating the subject.

35. A method for promoting tissue regeneration in a subject comprising implanting into the subject an effective amount of the scaffold of claim 1 into the subject, thereby treating the subject.

36. A method for promoting tissue regeneration in a subject comprising implanting into the subject an effective amount of the scaffold of claim 17 into the subject, thereby treating the subject.

37. A method for promoting bone formation in a subject, comprising implanting into the subject an effective amount of the scaffold of claim 10 into the subject, wherein the target specific growth factor comprises BMP-2.

38. A method for promoting bone formation in a subject, comprising implanting into the subject an effective amount of the scaffold of claim 26 into the subject, wherein the target specific growth factor consists essentially of BMP-2.

39. The method of claim 37 wherein the bone comprises cranial bone or spinal bone.

40. The method of claim 38 wherein the bone comprises cranial bone or spinal bone.

41. A method for promoting muscle repair in a subject, comprising implanting into the subject an effective amount of the scaffold of claim 10 into the subject, wherein the target specific growth factor comprises GDF11.

42. A method for promoting muscle repair in a subject, comprising implanting into the subject an effective amount of the scaffold of claim 26 into the subject, wherein the target specific growth factor consists essentially of GDF11.

43. A method for promoting cartilage repair in a subject, comprising implanting into the subject an effective amount of the scaffold of claim 10 into the subject, wherein the target specific growth factor comprises TGFB and IGF.

44. A method for promoting cartilage repair in a subject, comprising implanting into the subject an effective amount of the scaffold of claim 26 into the subject, wherein the target specific growth factor consists essentially of TGFB and IGF.

45. A method for promoting tendon repair in a subject, comprising implanting into the subject an effective amount of the scaffold of claim 10 into the subject, wherein the target specific growth factor comprises TGFB and GDF7.

46. A method for promoting tendon repair in a subject, comprising implanting into the subject an effective amount of the scaffold of claim 26 into the subject, wherein the target specific growth factor consists essentially of TGFB and GDF7.

47. A method for promoting regeneration of adipose tissue in a subject, comprising implanting into the subject an effective amount of the scaffold of claim 10 into the subject, wherein the target specific growth factor comprises insulin.

48. A method for promoting regeneration of adipose tissue in a subject, comprising implanting into the subject an effective amount of the scaffold of claim 26 into the subject, wherein the target specific growth factor consists essentially of insulin.

49. A method for promoting regeneration of nervous tissue in a subject, comprising implanting into the subject an effective amount of the scaffold of claim 10 into the subject, wherein the target specific growth factor comprises NGF.

50. A method for promoting regeneration of nervous tissue in a subject, comprising implanting into the subject an effective amount of the scaffold of claim 26 into the subject, wherein the target specific growth factor consists essentially of NGF.

51. The method of claim 35, wherein the scaffold is implanted in the subject in the region in need of repair.

52. The method of claim 36, wherein the scaffold is implanted in the subject in the region in need of repair.

53. A composition comprising:
(a) the scaffold of claim 1; and
(b) a pharmaceutically acceptable carrier.

54. A composition comprising:
(a) the scaffold of claim 17; and
(b) a pharmaceutically acceptable carrier.

55. A kit comprising the scaffold of claim 1 packaged with directions for its use.

56. A kit comprising the scaffold of claim 17 packaged with directions for its use.

57. A kit comprising the composition of claim 53 packaged with directions for its use.

58. A kit comprising the composition of claim 54 packaged with directions for its use.
